(19) United States
(12) Patent Application Publication
(10) Pub. No.: US 2018/0103947 A1
(43) Pub. Date: Apr. 19, 2018

(54) DEVICE FOR TENSIONING A SUTURE

(71) Applicant: Heartstitch, Inc., Fountain Valley, CA (US)

(72) Inventors: Anthony Nobles, Fountain Valley, CA (US); Christian Fankhänel, Ernstthal (DE); Robert Scheller, Fountain Valley, CA (US)

(21) Appl. No.: 15/844,351

(22) Filed: Dec. 15, 2017

Related U.S. Application Data
(63) Continuation-in-part of application No. 15/133,064, filed on Apr. 19, 2016, now abandoned.
(60) Provisional application No. 62/267,776, filed on Dec. 15, 2015.

(51) Int. Cl. A61B 17/04

(52) U.S. Cl. CPC A61B 17/0483; A61B 2017/00407

(57) ABSTRACT

A suture tensioning device that deters over-tensioning or under-tensioning of suture thread(s) is presented. The suture tensioning device includes an engagement portion and a tensioning portion. Optionally, the suture tensioning device also includes a detent portion. The tensioning portion has a tension limiter, which can be a protrusion, a wheel with a clutching mechanism, a spring, etc. A suture can be immobilized in the engagement portion of the suture tensioning device, and the tension can be increased or decreased by moving the tensioning portion. The tension limiter limits the movements of the tensioning portion so that the tension of the suture(s) can be set to a desirable level.

The application is a Continuation-In-Part of utility application Ser. No. 15/133,064, filed Apr. 19, 2016, which claims priority to provisional application Ser. No. 62/267,776, filed Dec. 15, 2015, and also claims priority to provisional application Ser. No. 62/434,816, filed Dec. 15, 2016.

### FIELD OF THE INVENTION

The field of the invention is systems, devices, and methods for tensioning a suture.

### BACKGROUND

The background description includes information that may be useful in understanding the present invention. It is not an admission that any of the information provided herein is prior art or relevant to the presently claimed invention, or that any publication specifically or implicitly referenced is prior art.

Surgeons frequently encounter the need to close incisions or wounds, or otherwise join tissue portions with a suture. After passing a suture thread through the tissue portions to be stitched together, a surgeon must tie and cinch the thread. When threads are tied in a region having restricted access, such as when deploying a suture through a catheter or trocar, tying the threads can be especially difficult. And regardless of where suturing is being performed, over-tensioning of the thread(s) can result in tearing the tissues, and under-tensioning can result in a failed suture. Additionally, the required tension for a suture thread can depend on various factors, such as the type of suture thread, the type of suturing procedure, and the type of suturing technique used.

There are many devices for holding and/or tying suture threads. For example, U.S. Patent application No. 2006/0282102 to Nobles discloses a knot-tying assist device having two suture receiving portions, which facilitates tying of the knot by regulating tension of the suture ends. Similarly, U.S. Pat. No. 7,762,584 to Lyons discloses a suture block, which includes a passageway with inwardly converging teeth that retains the suture inside the block. In another example, U.S. Patent application No. 2011/0190792 to Chu discloses an adaptor having a suture slit that is configured to maintain the tension of the suture. In Chu, the adaptor is configured to apply sufficient compression and/or friction force to the suture to maintain a tension on the suture during a medical procedure. Some suture retention devices hold multiple sutures simultaneously. For example, U.S. Pat. No. 7,762,584 to Morris discloses a suture retention device having a plurality of slots that maintain tension and orientation of the suture along the edge of a circular body of the retention device.

Others have tried to solve the over-tensioning/under-tensioning problem using mechanisms that directly tension the suture threads. For example, U.S. Pat. No. 3,650,274 to Edwards discloses an apparatus for controlling and distributing the tension in retention sutures. In Edwards, ends of a tied suture are placed within a slot in a positive locking capstan, and rotating the capstan increases or decreases the tension of the suture. In another example, U.S. Pat. No. 5,649,940 to Hart discloses a suture tensioning device that automatically adjusts a portion of a tension in the suture by a predetermined tension. In yet another example, U.S. Patent Application No. 2008/0097483 discloses a wheel disposed around the spool and having a suture wound there around, the wheel being movable between an engaged position in which the wheel rotates with the spool such that the biasing element biases the spool to maintain tension on the suture, and a disengaged position in which the spool rotates independent of the wheel to release the tension applied to the suture. In a last example, U.S. Pat. No. 6,471,715 to Weiss discloses a suture loop tension means having a manual control mechanism, operated by a rotating component, for pulling or releasing said second end for gradual application of a safe controlled tension and a means for measuring and displaying the tension in the suture integrally attached.

However, none of prior art references teaches an adjustable mechanism that allows a user to designate a proper tension level, indicates an extent of over-tensioning or under-tensioning of the suture relative to a designated tension level, and is convenient to use.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for improved suture tensioning devices and methods of adjusting desired tension levels.

### SUMMARY OF THE INVENTION

The inventive subject matter provides systems and methods for tensioning a suture.

One aspect of the invention includes suture tensioning devices that deter over-tensioning or under-tensioning of a suture. The device includes an engagement portion and a tensioning portion having a tension limiter. The engagement portion can be in various shapes, which include a V-shape, a curved V-shape, a Y-shape and a U-shape, a slit, a clamp, or a rail. In these embodiments, it is contemplated that the device has multiple engagement portions that are linearly or circularly oriented on the device.

In a preferred embodiment, the tensioning portion includes a pivot, which can include a clutching mechanism. Preferably, in this embodiment, the tension limiter in the tensioning portion is a spring configured to prevent the pivot from rotating more than a designated angle.

In other embodiments, the tensioning portion includes a wheel that is coupled with a clutching mechanism. In still other embodiments, the tensioning portion includes a translating mechanism that comprises two or more slidably mating portions. In these sliding embodiments, it is contemplated that the tensioning portion includes a protrusion as a tension limiter, which is disposed on a female one of the slidably mating portions.

In some embodiments, the device further includes a detent portion. The detent portion can be biased to close or open positions, depending on the shape of the detent portion. In one preferred embodiment, the detent portion has two arms that are tightly engaged with each other, which can be separated from each other by exerting pressure between the two arms.

Another aspect of the invention includes methods of preventing over-tensioning or under-tensioning of a suture. Some contemplated methods include a step of providing a suture tensioning device that has an engagement portion and a tension portion with a tension limiter, followed by steps of placing a portion of a suture in the engagement portion of the suture tensioning device, and operating a tension limiter until a predetermined or an adequate tension of the suture is achieved.

In preferred embodiments, the predetermined or adequate tension of the suture can be dynamically adjusted by a user based on one or more factors associated with the suturing operation. For example, an adequate tension could be adjusted based on the type of suture thread used, the type of suturing technique used, and the physical characteristics of the material to be sutured.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a schematic of a device for tensioning a suture thread.

FIG. 2 is a perspective view of a holding block for a suture tensioning device.

FIG. 3 is a perspective view of an alternative holding block for a suture tensioning device.

FIG. 4 is a perspective view of another alternative holding block for a suture tensioning device.

FIG. 5 is a perspective view of another alternative holding block for a suture tensioning device.

FIG. 6 is a perspective view of another alternative holding block for a suture tensioning device.

FIG. 7 is a perspective view of a rail used in a suture tensioning device.

FIG. 8A is a perspective view of a spring tension limiter for a suture tensioning device.

FIG. 8B is a perspective view of a operating mechanism for a spring tension limiter coupled to a suture block of a suture tensioning device.

FIG. 9 is a perspective view of an alternative spring tension limiter for a suture tensioning device.

FIGS. 10A-B are schematic views of yet another tension limiter for a suture tensioning device.

FIG. 11 is a schematic view of yet another spring tension limiter for a suture tensioning device.

FIG. 12 is a perspective view of still another holding block for a suture tensioning device.

FIG. 13 is a schematic view of a suture tensioning device with a tension indicator.

### DETAILED DESCRIPTION

The inventive subject matter provides apparatus, systems and methods for tensioning a suture and preventing over-tensioning or under-tensioning of the suture.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

One aspect of the inventive subject matter includes suture tensioning devices that deter over-tensioning or under-tensioning of a suture. Preferred suture tensioning device include an engagement portion and a tensioning portion having a tension limiter.

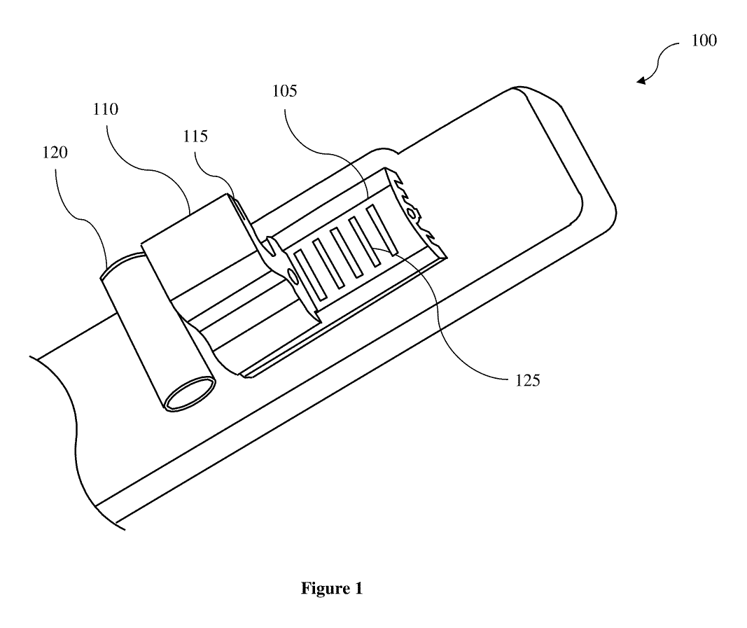

FIG. 1 shows one embodiment of a suture tensioning device 100. In this embodiment, the suture tensioning device 100 has a rail 105, a suture block 110, and a mechanism 120 that is configured to limit the tension of the suture. The mechanism 120 (e.g., button, switch, etc.) is preferably coupled with a tension limiter 125 (e.g., rail protrusions, a spring, a pivot, a wheel, etc.) such that operating mechanism 120 can initiate or stop the action of the tension limiter 125.

The suture block 110 has an engagement portion 115, in which a portion of the suture can be placed and stabilized (e.g., at least temporarily immobilized or has limited mobility, etc.). Preferably, the engagement portion 115 is shaped such that its inner portion is narrower than its outer portion. Thus, for example, it is generally preferred that the engagement portion is in a V-shape, a curved V-shape, a Y-shape or a U-shape. Yet, it is contemplated that the engagement portion 115 can be in any other suitable shape.

It is contemplated that the suture block 110 can comprise any suitable type(s) of material(s). For example, the suture block 110 can comprise metal materials (e.g., aluminum, copper, platinum, metal alloys, etc.), plastic materials (e.g., polypropylene, polyethylene, nylon, PVC or PTFE), metal materials (e.g., aluminum, copper, platinum, metal alloys, etc.), glass, or glass fiber. In a preferred embodiment, at least a portion of the suture block 110 comprises resilient materials (e.g., rubber, memory foam, plastic polymers, etc.).

Additionally, the suture block 110 further includes a detent portion (e.g., broader part of a “V”) that is coupled with an engagement portion 115 (e.g., narrower part of a “V”) such that the suture passes through the detent portion to reach the engagement portion. In some embodiments, the detent portion is biased to open such that it is essentially an opening for the suture to have an access to the engagement portion 115. In other embodiments, the detent portion is biased to close such that it allows the suture to pass through only upon a predetermined pressure exerted on the detent portion.

In a preferred embodiment, the suture block has a male component (e.g., flat surface, a round surface, a wheel, etc.) that is configured to slidably engage with a female component of a rail 105. As the suture block and the rail are slidably engaged, the suture block can move along the rail from the rail's one end to the other end. Thus, when the suture block retains a suture in the engagement portion 115, the tension of the suture can be at least partially modified by movement of the suture block 110.

In other contemplated embodiments (not shown) the suture block could have a female component that engages with a male component of a rail.

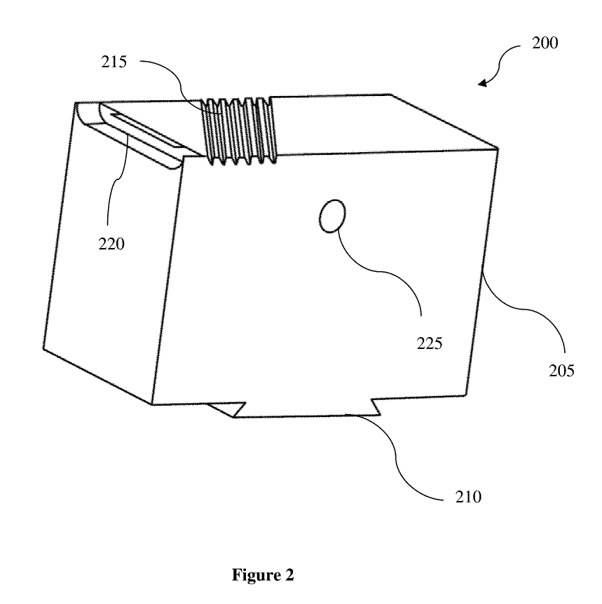

FIG. 2 shows a perspective view of an alternative suture block. Suture block 200 comprises box-shaped body 205, a male portion 210 to engage with a female portion of a rail (e.g., rail 105 in FIG. 1), a hole 225, and a hand or finger holding portion 220.

In the depicted embodiment, the suture block 200 has a plurality of suture thread engagement portions 215 that are preferably located in parallel with each other so that a plurality of suture threads can be immobilized on the suture block 200. Engagement portions 215 can be configured in any way that allows one or more suture threads to be individually separated and guidable through a concave channel. Preferably, engagement portions 215 are in the shape of a slit, a rail, a “U”, or a “V”. In some embodiments, the suture threads extending from a suture device can pass through a hole 225 on the body 205 before they are inserted into the engagement portion 215. When the suture threads are inserted into the engagement portion 215, and the suture block 200 is pushed or pulled, tension in all of the suture threads will be simultaneously regulated upon the movement of the suture block 200.

To aid the user in easily sliding the suture block 200, the suture block 200 includes a hand or finger holding portion 220.

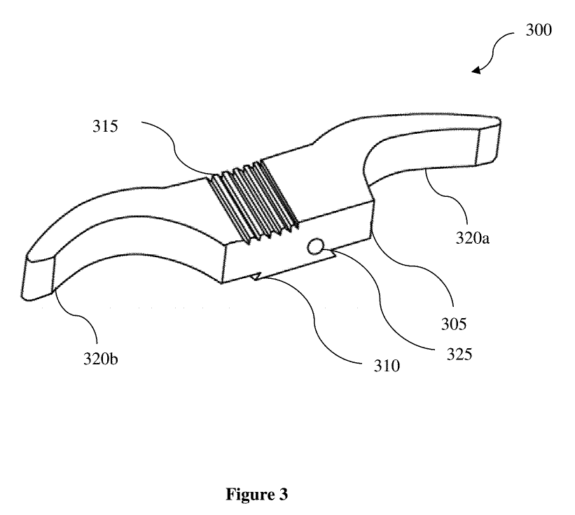

FIG. 3 shows a perspective view on another alternative suture block. Suture block 300 comprises a body 305, a male portion 310 that engages with a female portion of a rail (e.g., rail 105 in FIG. 1), engagement portions 315, two finger grip portions 320a and 320b, and a hole 325.

In the depicted embodiment, the suture block 300 once again has a plurality of slit-shaped (or V-shaped) engagement portions 315 that are preferably located in parallel with each other so that a plurality of sutures can be placed on the suture block 300. In some embodiments, the sutures from a suture device can pass through a hole 325 on the body 305 before they are placed on the engagement portion 315. When the suture threads are inserted into the engagement portion 315, and the suture block 300 is pushed or pulled, tension in all of the suture threads will be simultaneously regulated upon the movement of the suture block 300. In the embodiment shown in FIG. 3, the suture block also includes two finger-grip portions 320a, 320b to aid the user in easily sliding the suture block 300.

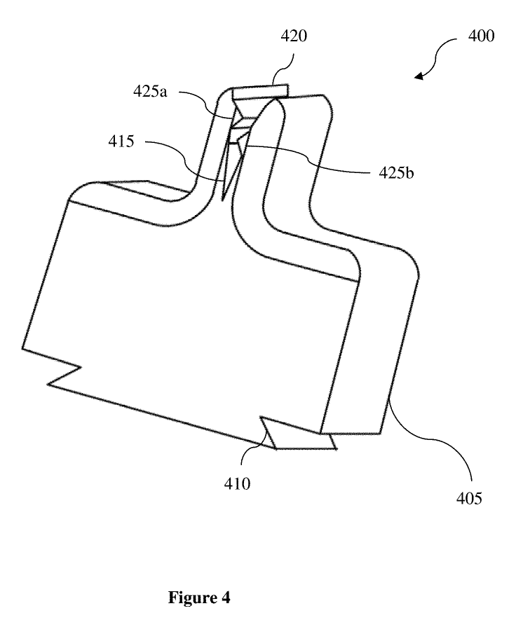

FIG. 4 is a perspective view of another alternative suture block. Suture block 400 comprises a body 405, a male portion 410 that engages with a female portion of a rail (e.g., rail 105 in FIG. 1), an engagement portion 415, a detent portion 420, and a plurality of teeth 425a and 425b.

In the depicted embodiment, the suture block 400 has a V-shaped suture thread engagement portion 415 in a protruded portion of body 405, and a detent portion 420 which is biased to an open configuration.

Preferably, the detent portion 420 has a plurality of teeth 425a and 425b that face one another and are either directly opposite of each other or offset. In an embodiment where the plurality of teeth 425a and 425b are face one another and are offset, the plurality of teeth 425a and 425b on the detent portion 420 provide a zigzagging path toward the engagement portion 415, such that a suture entered into the engagement portion 415 through the biased-to-open detent portion 420 is subject to frictional forces and cannot easily escape from the engagement portion 415.

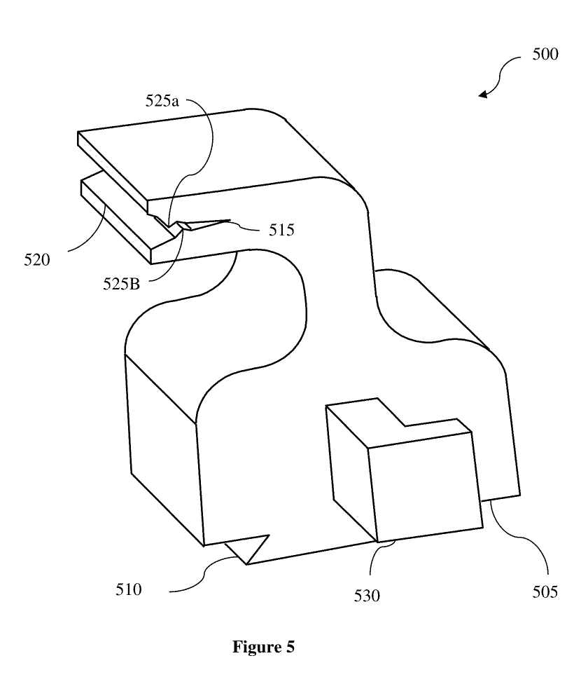

FIG. 5 is a perspective view of yet another alternative suture block. Suture block 500 has body 505, a male portion 510 that engages with a female portion of a rail (e.g., rail 105 in FIG. 1), a V-shaped suture thread engagement portion 515 that protrudes from the body 505, a detent portion 520 which is biased to open, a plurality of teeth 525a and 525b, and a finger-grip portion 530.

Preferably, the detent portion 520 has a plurality of teeth 525a, 525b that face one another and are either directly opposite of each other or offset. The plurality of teeth 525a and 525b on the detent portion 520 provide a zigzagging path toward the engagement portion 515, such that a suture entered into the engagement portion 515 through the biased-to-open detent portion 520 is subject to frictional forces cannot easily escape from the engagement portion 515. In the embodiment shown in FIG. 5, the suture block also includes a finger-grip portion 530 to aid the user in easily sliding the suture block 500.

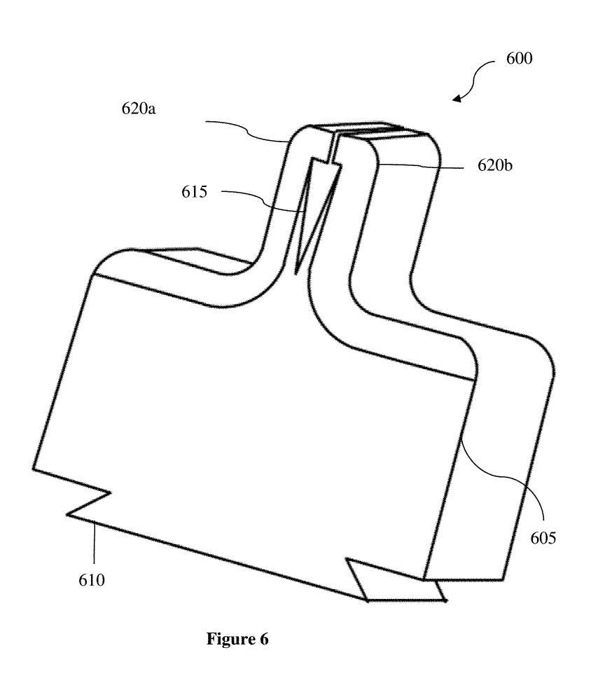

FIG. 6 is a perspective view of yet another alternative suture block. Suture block 600 has body 605, a male portion 610 to engage with a female portion of a rail (e.g., rail 105 in FIG. 1), an engagement portion 615, a clamp-type detent portion 620, and two arms 620a and 620b.

In the depicted embodiment, the suture block 600 has a V-shaped suture thread engagement portion 615 that protrudes from the body 605. The suture block 600 further includes a clamp-type detent portion 620 having two arms 620a and 620b, which at least partially meet with each other to allow the detent portion 620 biased to be in a closed configuration.

In a preferred embodiment, at least a portion of each arm (e.g., where the two arms meet) comprises one or more high friction materials (e.g., soft rubbers, plastic polymers, etc.). For example, the two arms 620a and 620b can be made of a resilient material, such as rubber, that springs back to a closed configuration after a force exerted to temporarily separate the two arms 620a and 620b, such that the detent portion 620 automatically closes after the suture passes through. Thus, the clamp-type detent portion 620 prevents the sutures in the engagement portion 615 from being inadvertently removed from the engagement portion 615.

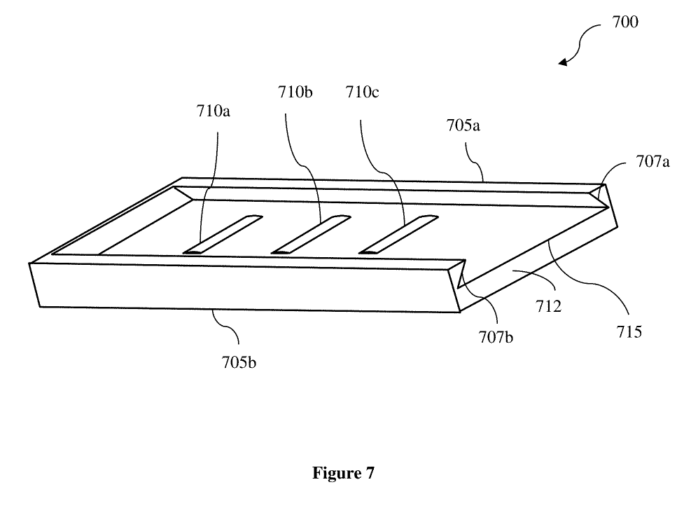

FIG. 7 is a perspective view of one embodiment of a rail. Rail 700 includes two side walls 705a and 705b, two inner surfaces 707a and 707b, protruded portions 710a-c, base 712, and an upper surface 715 of the base 712.

In the depicted embodiment, the two side walls 705a and 705b have inner surfaces 707a and 707b, respectively, which couple with the upper surface 715 to form a female portion within which an engaging portion of a suture block can slide. In some embodiments, at least one inner surface 707a or 707b is positioned at an angle of less than 90 degrees (e.g., 75 degree, 45 degree, etc.) with respect to the upper surface 715, such that an engaging portion of a suture block can be slidably disposed within and capable of movement substantially along one axis.

In much of the prior art, when a suture thread is engaged in an engagement portion of the suture block and the suture block is moved along a rail, the suture can be over-tensioned or under-tensioned. One class of solutions disclosed herein is to provide a tension limiter configured to limit tension in one or more suture threads by restricting translational movement of a suture block. Such restriction, for example, can be accomplished with stops, or with extension or compression springs.

As used herein, the term “spring” means a material that elastically returns to its original shape after it is compressed or expanded over a working range. Contemplated springs include helical, conical, volute and other coil springs, disc springs, leaf and other flat springs, and gas cartridge springs.

In FIG. 7, the tension limiter in the rail 700 includes protruded portions 710a, 710b, 710c, disposed on the upper surface 715. When a suture block (e.g., suture block 200 in FIG. 2) slidably moves along the rail 700, such movement of the suture block will be at least temporarily restricted when the male portion of the suture block encounters each of the protruded portions 710a-c. In a preferred embodiment, distances between adjacent protruded portions 710a and 710b, or between 710b and 710c, are less than 3 cm, preferably, less than 1 cm, and more preferably less than 0.2 cm.

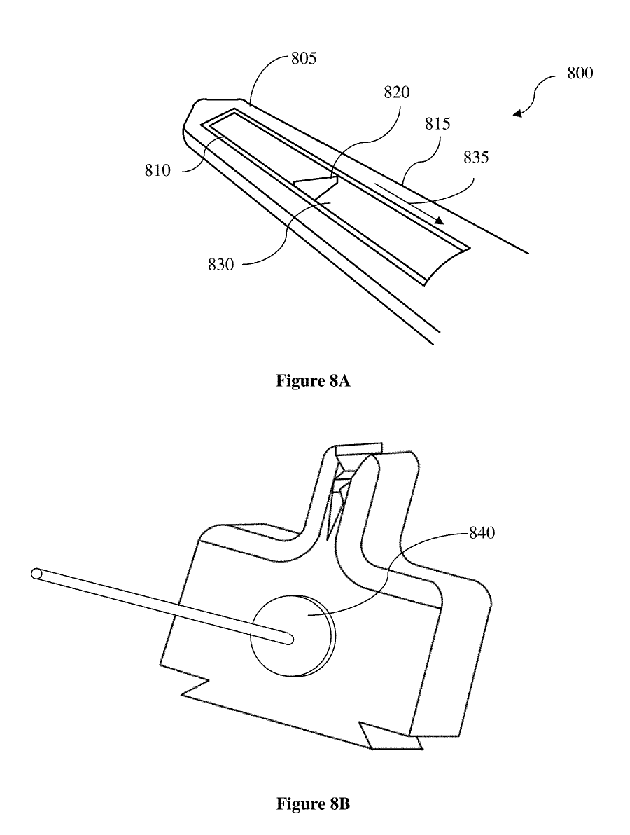

In FIG. 8, the tension limiter in the suture tensioning device 800 is a compression spring 830. The suture tensioning device 800 has a lower case 805 and an upper case 815 which are slidably engaged with each other over a rail 810. Within the upper case 815 is a suture block 820 and a compression spring 830. Having engaged one or more suture threads (not shown) in the engagement portion of the suture block 820, a user slides the upper case 815 over the lower case 805 in the direction of arrow 835, and then further tensions the suture thread(s) by pulling the suture block 820 in the direction of arrow 835. The compression spring 830 provides tactile feedback to the user, to assist in preventing the user from over-tensioning the suture thread(s).

In FIG. 8B, the suture block 820 in suture tensioning device 800 is coupled with a tension release mechanism 840.

In the depicted embodiment, it is contemplated that tension release mechanism 840 is a magnetic breakaway mechanism. For example, a magnetic connection between a magnetic affixed to suture block 820 and a magnet anchored to the lower case 805 breaks at a predetermined tension, thereby releasing tension on the suture threads by a predetermined amount in the event the tension exceeds a maximum threshold. In an additional embodiment, the operating mechanism 840 breaks away at a predetermined length associated with excessive tension on the suture threads.

It is further contemplated that the compression spring 830 could be coupled with an operating mechanism 840 (e.g., a switch, a button, a handle, one or more magnets, etc) that automatically releases compression in the compressed spring by at least 5 mm per operation to accommodate over-tensioning. For example, the operating mechanism can be a breakaway mechanism (e.g. magnetic connection designed to disengage at a predetermined tension or pull force, friction-based breakaway system, etc.) designed to release compression in the compressed spring by 5 mm when a suture is over tensioned. In another example, the operating mechanism can be an electronically controlled tensioning system using an electronic motor and a force meter to adjust the tension to the appropriate level based on the type of suture. Additionally or alternatively, the operating mechanism can be manually operated by the user to release the compression spring 830 by at least 5 mm per operation. For example, tension release mechanism 840 can have a ratcheting mechanism configured to have the pawl (e.g., portion engaging the teeth of the ratcheting gear) release a first tooth of the ratcheting gear at a predetermined tension level and move to the next tooth in the ratcheting gear after releasing the tension by a predetermined amount (i.e., 5 mm of slack to a line, reduction of tension by a particular number of newtons, etc.).

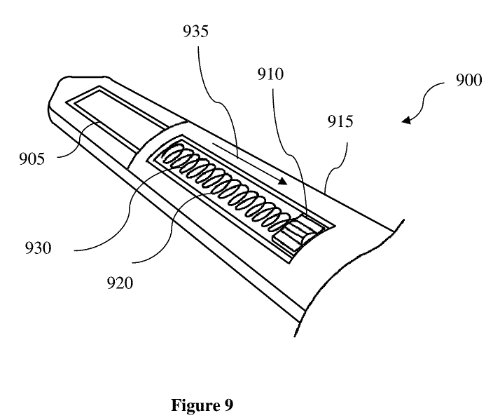

In FIG. 9, the tension limiter in the suture tensioning device 900 is an extension spring 930. The suture tensioning device 900 has a lower case 905 and an upper case 915 which are slidably engaged with each other over a rail 910. Within the upper case 915 is a suture block 920 and a compression spring 930. Having engaged one or more suture threads (not shown) in the engagement portion of the suture block 920, a user slides the upper case 915 over the lower case 805 in the direction of arrow 935, and then further tensions the suture thread(s) by pulling the suture block 920 in the direction of arrow 935. The extension spring 930 provides tactile feedback to the user, to assist in preventing the user from under-tensioning the suture thread(s).

It is further contemplated that the compression spring 930 could be coupled with an operating mechanism (e.g., a switch, a button, a handle, etc) (not shown) that automatically releases compression in the compressed spring by at least 5 mm per operation to accommodate over-tensioning. Additionally or alternatively, the operating mechanism can be manually operated by the user to release the compression spring 930 by at least 5 mm per operation.

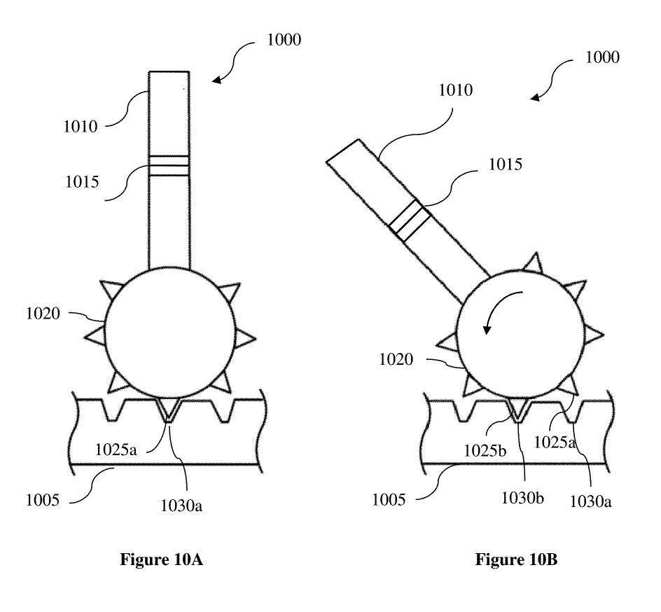

In FIGS. 10A-B, the tension limiter in the suture tensioning device is a lever 1000. The lever 1000 includes a handle portion 1010 having a suture engagement portion 1015 and a pivot portion 1020 with a plurality of teeth or other protrusions 1025a, 1025b extending from a curved perimeter (a ball shape, a circle shape, a wheel shape, etc.). The teeth or other protrusions 1025a, 1025b are preferably spaced along an engaging portion of the perimeter in at least every 30 degrees, preferably every 15 degrees, more preferably every 5 degrees, and engage with teeth or indentations 1030a, 1030b on a base 1005. For example, as depicted in FIG. 10A, when the lever is in a vertical position, protrusion 1025a is engaged with indentation 1030a. When the pivot portion 1020 is rotated in a counterclockwise direction (arrow) by tilting the handle portion 1010 of lever 1000 leftward, the protrusion 1025a is disengaged from the indentation 1030a, and the next protrusion 1025b is engaged with the next indentation 1030b of the base 1005. In that manner engagement of the various protrusions 1025 with corresponding indentations can tend to provide tactile feedback to the user, which can thereby assist in avoiding over- or under-tensioning of the suture thread(s).

It is also contemplated that in some embodiments, the pivot portion 1020 further includes a clutching mechanism (e.g., a gear, a spring, etc.) (not shown) that limits rotation of the pivot portion 1020 to less than 60 degrees, preferably to less than 45 degree, more preferably to less than 30 degree, most preferably less than 15 degree.

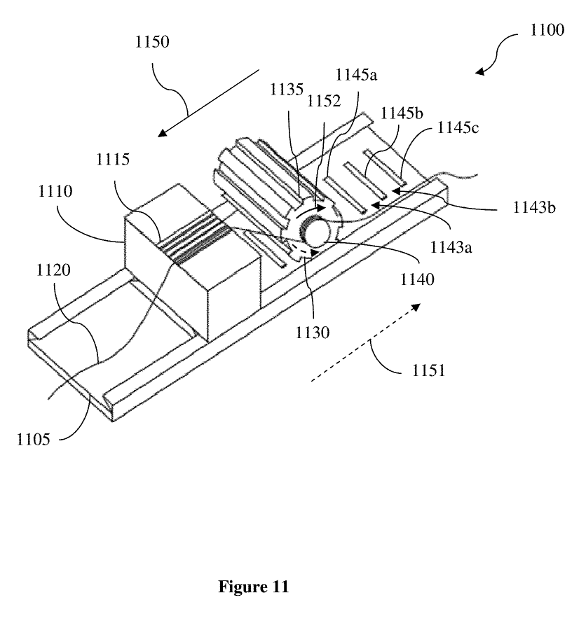

FIG. 11 is a perspective view of a suture tensioning system 1100, which includes a suture block 1110 having one or more suture thread engagement portions 1115. In a manner similar to those described with respect to FIGS. 2-7, the suture block 1110 is configured to slidably engage with a rail 1105 such that the suture block 1110 can move along the rail 1105.

In the embodiment depicted in FIG. 11, the tension limiter is a wheel 1130 having a suture wrapping portion 1140 and a plurality of teeth or other protrusions 1135. One or more suture threads 1120 is/are immobilized on the engagement portion 1115, and then wrapped around the suture wrapping portion 1140. Tension in the suture thread(s) can be regulated by rotating the wheel 1130 in the direction of arrow 1152, which translates the wheel 1130 along the rail 1105 in the direction of arrow 1151 While the wheel 1130 is moving along the rail 1105 to a proximal direction (solid arrow), the protrusion 1135 will be engaged with spaces 1143a, 1143b between any two of the rail protrusions 1145a, 1145b, 1145c. In the event that the suture thread(s) 1120 is/are over-tensioned, the wheel can be rotated in the direction of arrow 1150, which causes the wheel 1130 to translate along the rail 1105 in the direction of arrow 1150, which lessens tension in the suture thread(s) 1120 can be lessened.

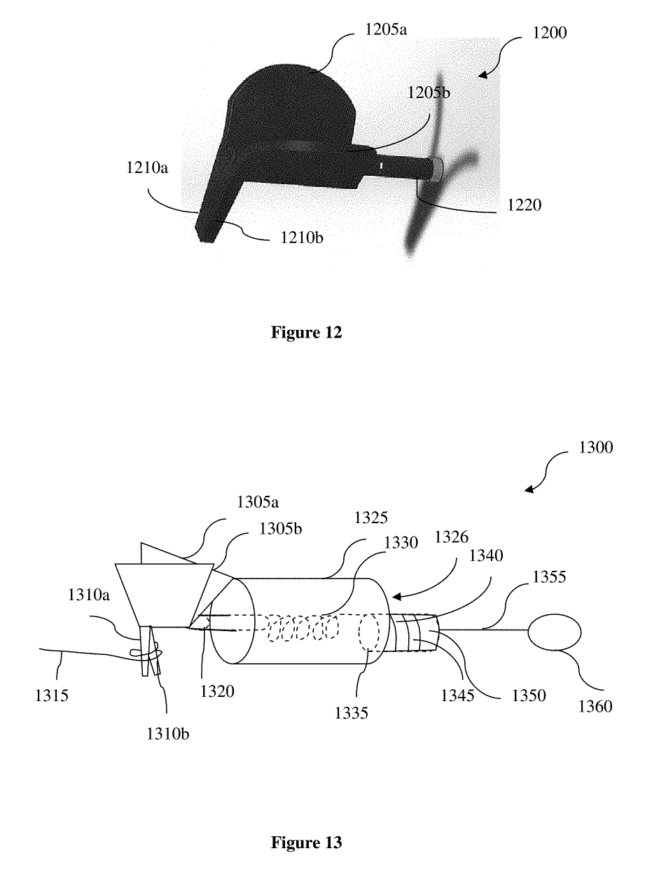

Another aspect of the inventive subject matter is the suture tensioning device coupled with a suture tension indicator. FIG. 12 shows one exemplary embodiment of a suture tensioning device 1200. The suture tensioning device 1200 includes a first suture holding portion 1210a and a second suture holding portion 1210b, which are coupled with a first finger contacting portion 1205a and a second finger contacting portion 1205b, respectively. In a preferred embodiment, the first suture holding portion 1210a and the second suture holding portion 1210b are hingeably coupled with each other such that the suture holding portions 1210a, 1210b, and finger contacting portions 1205a, 1205b form a shape of a clip. Thus, pressing finger contacting portions 1205a and 1205b towards each other increases the distance between the suture holding portions 1210a and 1210b by increasing the angle between the suture holding portions 1210a and 1210b. Preferably, the clip-shaped suture tensioning device 1200 allows the two suture holding portions 1210a and 1210b spread more than at least 90 degree, preferably at least 120 degree apart from each other so that the user can add sufficient tension to the suture when the sutures are wrapped around relatively larger area between two suture holding portions 1210a and 1210b. In this embodiment, a suture can be wrapped around the first suture holding portion 1210a and the second suture holding portion 1210b.

It is also contemplated that the suture tensioning device 1200 includes a locking mechanism (e.g., a spring-operated security scissor lock, a ring lock, etc.) at the suture holding portions 1210a, 1210b or at the finger contacting portions 1205a, 1205b, such that at least a portion of the suture can be tightly held between the first suture holding portion 1210a and the second suture holding portion 1210b.

FIG. 13 shows one exemplary embodiment of a suture tensioning system 1300 that includes a suture tensioning device 1305, a first suture holding portion 1310a, a second suture holding position 1310b, a suture 1315, a connector 1320, an elongated body 1325, a lumen 1326, a spring 1330, and a tension indicator 1335.

Suture tensioning system 1300 includes a suture tensioning device 1305. The suture tensioning device 1305 includes a first suture holding portion 1310a and a second suture holding portion 1310b, which are coupled with a first finger contacting portion 1305a and a second finger contacting portion 1305b, respectively. Similar to the suture tensioning device 1200 of FIG. 12, the first suture holding portion 1310a and the second suture holding portion 1310b are sized and dimensioned so that a suture 1315 can be wrapped around the suture holding portions 1310a, 1310b and preferably at least a portion of the suture 1315 can be tightly held between the suture holding portions 1310a, 1310b.

The suture tensioning system also includes an elongated body 1325 having a lumen 1326. The elongated body 1325 includes a spring 1330, a connector 1320, and a tension indicator 1335 in the lumen. As used herein, a lumen 1326 refers an inside space of a structure. In some embodiments, the shape of the lumen 1326 depends on the shape of the elongated body 1325. For example, the elongated body 1325 can be a tubular shape, a twisted tubular shape, a cuboid shape, or a triangular prism shape, and the shape of the lumen 1326 can be shaped to match the elongated body 1325. In other embodiments, the shape of the lumen 1326 can be independent from the shape of the elongated body 1325. For example, where an elongated body 1325 has tubular shape, the lumen 1326 could have a cuboid shape, or vice versa.

The spring 1330 is coupled with the suture tensioning device 1305 at one end (e.g., distal end, etc.) via a first connector 1320 (e.g., a rod, a cord, a string, etc.) and with the tension indicator 1335 at the other end (e.g., proximal end, etc.). The tension indicator 1335 is also coupled with a user handle 1360 via a second connector 1355 (e.g., a rod, a cord, a string, etc.). In a preferred embodiment, the tension indicator 1335 comprises a plurality of portions that are color-coded in different colors. For example, the tension indicator 1335 includes a first portion 1340 that is red-colored, a second portion 1345 that is green-colored, and a third portion 1350 that is gray-colored. In some embodiments, the area of the portions 1340, 1345, 1350 are predetermined area (e.g., predetermined length, area, height, etc.) and permanently colored (e.g., painted, etc.). In other embodiments, the area of the portions 1340, 1345, 135 can be changed depending on the type and strength of the spring used. For example, in these embodiments, the tension indicator 1335 comprises a plurality of LED that can provide gradients of colors of lights in different areas on the surface of the tension indicator 1335. In those embodiments, the location of the areas that are color-coded can be moved as needed. For example, area 1 in the tension indicator 1335 can have LEDs in green color, but the color can be changed to red if different type of spring is used. Also, area 1 in the tension indicator 1335 in green color can later be split into two areas with two different colors (e.g., half in green LED and half in red LED, etc.)

When the sutures 1315 are coupled with the suture tensioning device 1305, the user can pull and/or push the user handle 1360 to modulate the tension of the suture to its desired level. In some embodiments, the spring rate (e.g., the amount of weight that is needed to compress or pulls a spring one inch) of the spring 1330 determines the pre-determined suture tension. Thus, in these embodiments, when the user begins to pull the user handle 1360, the third portion 1350 of the indicator 1335 exposes outside of the elongated body 1325 and shows up to the user first, which may indicate that suture 1315 is under-tensioned. When the user pulls the user handle 1360 further, the second portion 1345 of the indicator 1335 shows up to the user, which may indicate that suture 1315 is tensioned in a desired level. If the user pulls the pulls the user handle 1360 still further, the first portion 1340 of the indicator 1335 shows up to the user, which may indicate that suture 1315 is over-tensioned in a desired level. Then, the user can release or push the user handle 1360 until the second portion 1345 of the indicator 1335 shows up to the user again.

It is contemplated that the indicator 1335 can be adjusted by the user to calibrate the desired tension level based on the factors associated with the suturing operation. For example, a user can replace the spring 1330 to adjust the spring rate in order to adjust the tactile feedback to the user that indicates a desired tension level. In another example, a user can calibrate the desired tension level by adjusting the placement of an indicator of a desired tension level such that the indicator causes a user to compress the spring by an amount that corresponds to the desired tension level without changing the spring rate. However, the present invention contemplates the use of any method of limiting tension known in the art.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A suture tensioning device that deters over-tensioning or under-tensioning of a suture, the device comprising:
a base;
a block having at least a suture engagement portion, and configured to move relative to the base;
the suture engagement portion includes a first slot configured to hold a suture portion, and having a width that varies from greater than a width of the suture to narrower than the width of the suture; and
a spring configured to cooperate with the block, such when the suture portion is held by the first slot, a tension applied to the suture is proportional to an amount to which the spring is compressed or extended by movement of the block with respect to the base.

2. The device of claim 1, wherein the first slot includes a detent portion that is biased to close.

3. The device of claim 2, wherein the detent portion comprises first and second arms that are tightly engaged with each other.

4. The device of claim 3, wherein the first and second arms are separated from each other upon a pressure exerted between the first and second arms.

5. The device of claim 1, further comprising a detent portion, wherein the detent portion is biased to open.

6. The device of claim 1, wherein the spring is a coil spring.

7. The device of claim 1, wherein the first slot is V-shaped.

8. The device of claim 1, wherein the block includes at least a second slot parallel to the first slot.

9. The device of claim 1, wherein having a base has a race, and the block is configured to slide within the race.

10. The device of claim 1, wherein the block is configured to pivot more than 45 degree with respect to the base
